(19) United States
(12) Patent Application Publication
(10) Pub. No.: US 2010/0172869 A1
(43) Pub. Date: Jul. 8, 2010

(54) METHOD FOR TREATING MULTIPLE SCLEROSIS

(75) Inventor: Lorianne K. MASUOKA, Oakland, CA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS VACCINES AND DIAGNOSTIC, INC., EMERYVILLE, CA (US)

(21) Appl. No.: 12/641,856

(22) Filed: Dec. 18, 2009

Related U.S. Application Data
(63) Continuation of application No. 11/857,245, filed on Sep. 18, 2007, now abandoned.
Application No. 11/857,245 is a continuation of application No. 10/246,932, filed on Sep. 18, 2002, now abandoned.
(60) Provisional application No. 60/322,933, filed on Sep. 18, 2001.

(51) Int. Cl. A61K 38/21; A61P 25/00

(52) U.S. Cl. 424/85.6

(57) ABSTRACT

Methods for treating multiple sclerosis (MS) and clinically isolated syndromes suggestive of MS are provided. The methods comprise administering a therapeutically effective dose of interferon-beta (IFN-beta) to a subject in need thereof, where the dose is administered intramuscularly with a dosing frequency of two- to three-times per week.

### CROSS-REFERENCE TO RELATED APPLICATIONS

This application is a continuation of co-pending U.S. patent application Ser. No. 10/246,932, filed Sep. 18, 2002, which claims the benefit of U.S. Provisional Application Ser. No. 60/322,933 filed Sep. 18, 2001, the contents of both of which are hereby incorporated herein in their entirety by reference.

### FIELD OF THE INVENTION

The present invention is directed to new treatment regimens for multiple sclerosis (MS) and clinically isolated syndromes suggestive of MS.

### BACKGROUND OF THE INVENTION

Multiple sclerosis (MS) is a severe, chronic disabling disease that affects approximately 1 out of every 1,600 people. The majority of the affected individuals develop symptoms as young adults between 20 and 40 years of age, with roughly 60% of the cases occurring in women. The disease is characterized by neuron deterioration in the central nervous system (CNS) with the associated loss of the insulating myelin sheath from around the axons of the nerve cells, referred to as demyelination. The disease presents itself in the white matter of the brain and spinal cord as a number of sclerotic lesions or plaques (Prineas (1985) Demyelinating Diseases, Elsvevier: Amsterdam; Raine (1983) Multiple Sclerosis, Williams and Wilkins: Baltimore; Raine et al. (1988) J. Neuroimmunol. 20:189-201; and Martin (1997) J. Neural Transmission (Supply) 49:53-67). The characteristic MS lesion is inflamed, exhibits axonal demyelination, axonal degeneration, and is found around small venules. These characteristics typically evolve early in plaque development and are hypothesized to occur as a result of a breakdown in the blood-brain barrier (BBB). As a consequence of BBB breakdown, infiltrates consisting of various lymphocytes and macrophages enter the brain or spinal cord. This inflammatory infiltrate ultimately leads to axonal degeneration and scar tissue formation, and in many instances, is associated with incomplete remyelination (Martin (1997) J. Neural Transmission (Suppl.) 49:53-67). Further, it is hypothesized that this apparent immunologic attack targets not only the myelin sheath, but also the oligodendrocytes imperative to CNS myelin production. As a result, not only is the nerve-insulating myelin damaged, but the ability of oligodendroglial cells to repair damaged myelin is seriously compromised (Scientific American 269 (1993):106-114). Development of multiple areas of scar tissue (sclerosis) along the covering of the nerve cells slows or blocks the transmission of nerve impulses in the affected area, resulting in the development of the symptoms characteristic of MS. These symptoms include pain and tingling in the arms and legs; localized and generalized numbness, muscle spasm and weakness; difficulty with balance when standing or walking; difficulty with speech and swallowing; cognitive deficits; fatigue; and bowel and bladder dysfunction.

Approximately half of the people with this disease suffer from relapsing-remitting MS. In these cases, the afflicted individual experiences repeated unpredictable attacks, due to episodes of inflammation, axonal demyelination, axonal degeneration, and development of glial scar tissue. These attacks are separated by periods of remission, during which the symptoms stabilize or diminish. Acute neurological deficits occur with each attack, and in many cases, the accumulation of residual deficits as a result of these attacks eventually leads to worsening disability and impairment in quality of life. Approximately 30-40% of the afflicted population have chronic progressive MS (either primary or secondary) in which neurological deterioration occurs in the absence of clinically apparent attacks.

Recently, immunomodulatory therapy with interferon-beta (IFN-beta) has proven to be successful in reducing the severity of the underlying disease in patients with relapsing-remitting MS. FDA-approved IFN-beta therapies for the treatment of relapsing-remitting MS in the United States include interferon beta-1a (marketed as Avonex®, available from Biogen. Inc.) and interferon-beta-1b (marketed as Betaseron®, available from Chiron Corporation). Both of these therapeutic agents are partially effective in reducing the frequency and severity of relapses, slowing the rate of disease progression, or reducing the degree of brain inflammation as measured by a variety of magnetic resonance imaging (MRI) techniques. Both of these therapies are systemic, requiring injections.

The IFN-beta-1a in Avonex® is the glycosylated, native human sequence that has been produced in Chinese Hamster ovary cells using recombinant DNA technology. The IFN-beta-1b in Betaseron® is the unglycosylated, serine 17-substituted, native human sequence that has been recombinantly produced in Escherichia coli. The approved regimen for Avonex® is once-weekly intramuscular injection of 6 MIU (30 μg). Betaseron® is administered subcutaneously, 8 MIU (250 μg), every other day. Rebif® (available from Serono. Inc.) is a third IFN-beta medication for use in treatment of relapsing-remitting MS and is currently awaiting US FDA approval. The European Commission-approved protocol for Rebif®, which also contains IFN-beta-1a manufactured from Chinese Hamster ovary cells, is three times weekly subcutaneous injections of 12 MIU (44 ucg) or 6 MIU (22 ucg) for patients not tolerating the higher dose.

At this time, no interferons are approved for use in secondary progressive MS in the United States (US), although Biologic License Applications (BLA) for Betaseron® and Rebif® using the same dosing regimens as those approved for relapsing-remitting MS, are under review by the US FDA. Betaseron® is approved for use in the treatment of secondary progressive MS in the European Union (EU) for those patients still experiencing relapses. For this indication, Betaseron® is administered subcutaneously, 8 MIU, every other day. Interferons are not yet approved for use in the treatment of primary progressive MS or clinically isolated syndromes suggestive of MS (also known as early onset MS or monosymptomatic MS) in the US or EU, although a BLA for Avonex® for use in the treatment of monosymptomatic MS is under review by the US FDA.

Clinical efficacy of these IFN-beta medications is dependent upon dose and dose frequency. In 1993. Betaseron® became the first beta interferon to be approved for use in the US for the treatment of relapsing-remitting MS. The pivotal clinical trial demonstrated that Betaseron® reduces the rate of attacks by approximately 31% in a two year period (IFNB Multiple Sclerosis Study Group (1993) Neurology 43(4):655-661). In 1996, Avonex® was also approved for use in the US for the treatment of relapsing-remitting MS. This pivotal clinical trial demonstrated that Avonex® reduces the rate of attacks by approximately 18% over two years (prescribing information for Avonex®). Although the publication of the results of this study indicated a roughly 32% reduction in exacerbation rate (Multiple Sclerosis Collaborative Research Group (1996) Ann. Neural. 39(3):285-294), data validated by the US FDA appear to indicate the possibility that Avonex® is somewhat less efficacious than Betaseron® for the reduction of relapses in patients with relapsing-remitting MS. It is more difficult to compare the effect of these interferons on progression rate, as the methods employed for measuring progression were somewhat different in the two studies.

One pharmacology study points to a potential explanation for why Avonex® may be less efficacious than Betaseron® in treating relapses (Williams and Witt (1998) J. Interferon and Cytokine Res. 19:967-975). This study compared the pharmacodynamic effect of once-weekly intramuscular Avonex® versus every-other-day subcutaneous Betaseron® in healthy volunteers. The binding of IFN-beta to the type I inteferon receptor results in the induction of certain biological response markers such as neopterin, β₂ microglobulin, and IL-10. All these markers showed a greater induction following Betaseron® administration (as measured by area under the curve over the entire 7 day observation period) than following Avonex® administration. The serum neopterin levels appeared to fall significantly 48 hours after administration of Avonex®, and were dramatically reduced (>50%) by 72 hours. Serum neopterin levels were sustained for the entire 7-day observation period following administration of Betaseron® every other day.

A recently completed comparative study of Rebif® (IFN beta-1a) versus Avonex® indicates that total dose may also play a role in overall clinical efficacy (2001 World Congress of Neurology, London). Preliminary results of this study indicate that Rebif® 12 MIU (44 ucg) subcutaneously three times per week is more effective in reducing the rate of relapse than Avonex® 6 MIU (30 ucg) intramuscularly once weekly. However, Avonex® 12 MIU (60 ucg) weekly was not shown to be superior to Avonex® 6 MIU (30 ucg) weekly (Biogen website) underscoring the potential importance of dosing frequency us well as total dose.

In addition, the route of administration of these medications influences their side effect profiles, making choice of a preferred medication more complex. Two IFN-beta medications, Betaseron® and Rebif®, are administered via multiple subcutaneous injections weekly. Both medications are associated with a high incidence (up to 85%) of injection site reactions, and the most serious type of injection site reaction, skin necrosis, occurs in approximately 5% of patients using either product. Avonex®, which is also an IFN beta-1a product but is administered intramuscularly, differs significantly with respect to injection site reactions. The overall incidence of these reactions is substantially lower for this product, and injection site necrosis rarely if ever occurs.

Although it is unclear whether route of administration plays a role in liver function abnormalities, the reported incidence of elevated liver transaminases appears lower for the intramuscularly administered Avonex® than for the subcutaneously administered Betaseron® and Rebif®. Similarly, the incidence of neutralizing antibodies is substantially lower for Avonex® than for Rebif® or Betaseron®. It unclear however, whether frequency of administration or total protein delivered plays a role in this difference (with fewer weekly injections and lower protein delivery for Avonex®).

Clearly additional treatment regimens are needed to provide improved efficacy and safety of interferon-beta for use in reducing disease severity in patients with multiple sclerosis.

### SUMMARY OF THE INVENTION

Methods for treating a subject suffering from multiple sclerosis (MS) and clinically isolated syndromes suggestive of MS are provided. The methods comprise administering to the subject a therapeutically effective dose of interferon-beta (IFN-β) or biologically active variant thereof two times per week or three times per week, where administration is by intramuscular injection. Interferon-beta or biologically active variant thereof is administered in the range of about 3 MIU to about 30 MIU per injection. The dosing regimens of the present invention maximize clinical efficacy of intramuscular injection of IFN-beta for treatment of MS and reduce adverse side effects such as injection site reactions frequently associated with clinically acceptable subcutaneous injection treatment regimens.

### BRIEF DESCRIPTION OF THE DRAWINGS

FIG. 1 sets forth the amino acid sequence for mature human interferon-beta (SEQ ID NO:1).

FIG. 2 sets for the amino acid sequence for the mature human interferon-beta mutein IFN-beta_{Ser17} (SEQ ID NO:2).

### DETAILED DESCRIPTION OF THE INVENTION

The present invention is directed to methods for treating multiple sclerosis (MS) and clinically isolated syndromes suggestive of MS. The methods comprise administering a therapeutically effective dose of interferon-beta (referred to as IEN-beta or IFN-β) or biologically active variant thereof to a patient in need of treatment, where the dose is administered intramuscularly two- to three-times weekly as noted below. The methods are beneficial in the treatment of patients suffering from various clinically recognized forms of multiple sclerosis, including relapsing-remitting MS, all forms of progressive MS including but not necessarily limited to primary and secondary progressive MS, and progressive-relapsing MS, as well as clinically isolated syndromes suggestive of MS.

By “relapsing-remitting” MS is intended a clinical course of MS that is characterized by clearly defined, sporadic acute attacks (exacerbations or relapses), during which existing symptoms become more severe and/or new symptoms appear. These attacks, lasting anywhere from days to months, are followed by partial recovery, or full recovery and remission. The length of time between these sporadic attacks may be months or years, during which time microscopic lesions, axonal loss, and scar formation still proceed. Relapsing-remitting MS is the most common beginning phase of MS, with about 50% of the cases having progression within 10 to 15 years, and another 40% within 25 years of onset.

By “secondary-progressive” MS is intended a clinical course of MS that initially is relapsing-remitting and then becomes progressive at a variable rate independent of relapses, possibly interspersed with relapses and remissions. As recovery from attacks is less and less complete with disease progression, physical and mental impairment increase. The actual clinical attacks become less well defined, are not as acute as in relapsing-remitting MS, and remissions become less apparent. Concomitant with this phase of MS, CNS tissue damage is cumulative, as evidenced by MRI analysis. Though patients experiencing this type of MS can continue to experience inflammatory attacks or exacerbations, eventually the attacks and periods of remission diminish, with the disease taking on the characteristic decline observed with primary-progressive MS.

By “primary-progressive” MS is intended a clinical course of MS that is characterized from the beginning by progressive disease, with no plateaus or remissions, or an occasional plateau and very short-lived, minor improvements. As the disease slowly progresses, the patient experiences difficulty walking, motor skills steadily decline, and disabilities increase over many months and years, generally in the absence of those distinct inflammatory attacks characteristic of relapsing-remitting MS.

By “progressive-relapsing” MS is intended a clinical course of MS that shows permanent neurological deterioration from the onset of the disease, but with clear, acute exacerbations or relapses that look like relapsing-remitting MS. For these patients, lost functions generally never return. Left untreated, this type of MS has a high mortality rate.

Clinically isolated syndromes suggestive of MS include, but are not limited to, early onset multiple sclerosis and monosymptomatic MS. For purposes of the present invention, the term “multiple sclerosis” is intended to encompass each of these clinical manifestations of the disease and clinically isolated syndromes suggestive of MS unless otherwise specified.

The methods of the present invention represent new dosing regimens for use of IFN-beta for multiple sclerosis. These new regimens address the shortcomings of heretofore known clinically accepted protocols using interferon-beta as described above. Although these clinically accepted protocols are partially effective in reducing the frequency and severity of relapses, slowing the rate of disease progression, or reducing the degree of brain inflammation as measured by a variety of MRI techniques, they vary in efficacy and tolerability. Hence, protocols requiring subcutaneous injection of IFN-beta-1b every other day (i.e., Betaseron® as approved for MS by FDA) or subcutaneous injection of IFN-beta-1a (Rebif® as approved for MS by the EC) three times per week appear to be more efficacious than protocols requiring intramuscular injection of INF-beta-1a once per week (i.e., Avonex® as approved for MS by FDA). However, the subcutaneous injection protocols are associated with a high incidence of injection site reactions, including skin necrosis, as noted above. In contrast, the approved protocol requiring an intramuscular route, though less efficacious, has a substantially lower overall incidence of injection site reactions.

The dosing regimens disclosed herein provide for improved efficacy of intramuscular injection of IFN-beta in treating disease progression and/or symptoms associated with MS without compromising the beneficial safety profile associated with this administration route. Without being bound by theory, it is believed that maximal clinical efficacy and safety profile depend less upon the type of IFN-beta (for example, IFN-beta-1a versus IFN-beta 1b) than on the route of administration, dose, and dosing frequency. The dosing regimens disclosed herein are thus designed to both maximize clinical efficacy and reduce adverse effects such as injection site reactions and hepatotoxicity. Clinical efficacy is maximized by increasing the number of therapeutically effective doses of IFN-beta or biologically active variant thereof administered each week, using the administration route providing the superior safety profile, i.e., intramuscular injection.

In accordance with these new dosing regimens, a therapeutically effective dose of INF-beta or biologically active variant thereof is administered intramuscularly, two- to three-times weekly, to a subject suffering from multiple sclerosis. Preferably the therapeutically effective dose is delivered by intramuscular injection (IM) into the large muscles of the thigh, upper arm, or hip.

A “therapeutically effective dose” of IFN-beta or biologically active variant thereof is a dose of IFN-beta or biologically active variant thereof that, when administered intramuscularly in accordance with a dosing frequency of two- to three-times weekly, provides for treatment of multiple sclerosis. By “treating” or “treatment” of multiple sclerosis is intended the methods of the present invention result in an improvement in the disease in a patient undergoing the dosing regimens of the present invention, and/or an improvement in the symptoms associated with the disease. Thus, when a patient suffering from multiple sclerosis undergoes treatment in accordance with the methods of the present invention, treatment can result in the prevention and/or amelioration of disease symptoms noted below, disease severity, and/or periodicity or recurrence of the disease, that is, the methods can result in lengthening the time period between episodes in which symptoms flare, and/or can suppress the ongoing immune or autoimmune response associated with the disease, which, left untreated, enhances disease progression and disability.

Factors influencing the amount of IFN-beta or biologically active variant thereof that constitutes a therapeutically effective dose include, but are not limited to, the severity of the disease, the history of the disease, and the age, health, and physical condition of the individual undergoing therapy. Generally, a higher dosage of this therapeutic agent is preferred as tolerated.

In accordance with the methods of the present invention, a therapeutically effective dose of IFN-beta or biologically active variant thereof is in the range of about 3 MIU to about 30 MIU per injection, about 3.5 MIU to about 25 MIU per injection, preferably about 4 MIU to about 20 MIU per injection, more preferably about 4.5 MIU to about 17 MIU per injection, still more preferably about 5 MIU to about 15 MIU per injection, most preferably about 6 MIU to about 12 MIU per injection. Thus, in one embodiment, the therapeutically effective dose of IFN-beta or biologically active variant thereof to be administered intramuscularly per injection according to the preferred dosing schedule is about 3 MIU to about 5 MIU, about 5 MIU to about 7 MIU, about 7 MIU to about 9 MIU, about 9 MIU to about 11 MIU, about 11 MIU to about 13 MIU, about 13 MIU to about 15 MIU, about 15 MIU to about 17 MIU, about 17 MIU to about 19 MIU, about 19 MIU to about 21 MIU, about 21 MIU to about 24 MIU, about 24 MIU to about 27 MIU, or about 27 MIU to about 30 MIU, depending upon the dosing frequency and severity of the disease in the patient undergoing treatment. The average human is approximately 1.7 m². Thus, the therapeutically effective dose on a per m² basis to be administered to a subject per injection is equivalent to about 1.76 MIU/m² to about 17.6 MIU/m², preferably within the range of about 3.5 MIU/m² to about 7.0 MIU/m².

In order to maximize clinical efficacy and reduce adverse effects associated with injection, the therapeutically effective dose of IFN-beta or biologically active variant thereof is administered intramuscularly with a dosing frequency of two- to three-times per week, such as two times per week or three times per week, preferably two times per week (i.e., twice weekly). This dosing regimen is continued for as long as is required to achieve the desired effect, that is, for example, prevention and/or amelioration of the disease, symptoms associated with the disease, disease severity, and/or periodicity of the recurrence of the disease, as noted above. In one embodiment, the dosing regimen is continued for a period of up to one year to indefinitely, such as for one month to 30 years, about three months to about 20 years, about 6 months to about 10 years. Because of the reduced side effects associated with this treatment protocol, the patient can remain on this dosing regimen indefinitely until the desired objective is achieved.

Thus, where a patient suffering from relapsing-remitting MS undergoing therapy in accordance with the previously mentioned dosing regimens exhibits a partial response, or a relapse following a prolonged period of remission, subsequent courses of therapy in accordance with the methods of the present invention may be needed. Thus, subsequent to a period of time off from a first treatment period, a patient may receive one or more additional treatment periods, each comprising intramuscular administration of a therapeutically effective dose of IFN-beta or biologically active variant thereof two- to three-times weekly for as long as necessary to bring the disease back into remission or to ameliorate disease symptoms.

Symptoms of MS that are prevented, ameliorated, or treated when a patient undergoes therapy in accordance with the methods of the present invention include: weakness and/or numbness in one or more extremities; tingling of the extremities and tight band-like sensations around the trunk or limbs; tremor of one or more extremities; dragging or poor control of one or both legs to spastic or ataxic paraparesis; paralysis of one or more extremities; hyperactive tendon reflexes; disappearance of abdominal reflexes; Lhermitte's sign; retrobulbar or optic neuritis; unsteadiness in walking; increased muscle fatigue; brain stem symptoms (diplopia, vertigo, vomiting); disorders of micturition; hemiplegia; trigeminal neuralgia; other pain syndromes; nystagmus and ataxia; cerebellar-type ataxia; Charcot's triad; diplopia; bilateral internuclear opthalmoplegia; myokymia or paralysis of facial muscles; deafness; tinnitus; unformed auditory hallucinations (because of involvement of cochlear connections); transient facial anesthesia or of trigeminal neuralgia; bladder dysfunction euphoria; depression; fatigue; dementia, dull, aching pain in the low back; sharp, burning, poorly localized pains in a limb or both legs and girdle pains; abrupt attacks of neurologic deficit; dysarthria and ataxia; paroxysmal pain and dysesthesia in a limb; flashing lights; paroxysmal itching; and/or tonic seizures, taking the form of flexion (dystonic) spasm of the hand, wrist, and elbow with extension of the lower limb. A patient having MS may have one or more of the symptoms associated with MS and one or more can be ameliorated by the dosing regimens of the present invention.

The dosing regimens disclosed herein can also block or reduce the physiological and pathogenic deterioration associated with MS, e.g., inflammatory response in the brain and other regions of the nervous system, breakdown or disruption of the blood-brain barrier, appearance of lesions in the brain, tissue destruction, demyelination, autoimmune inflammatory response, acute or chronic inflammatory response, neuronal death, and/or neuroglial death. Beneficial effects of the dosing regimens of the present invention include, e.g., preventing the disease, slowing the onset of established disease, ameliorating symptoms of the disease, reducing the annual exacerbation rate (i.e., reducing the number of episodes per year), slowing the progression of the disease, or reducing the appearance of brain lesions (e.g., as identified by MRI scan), and postponing or preventing disability including cognitive decline, loss of employment, hospitalization, and finally death. The episodic recurrence of the particular type of MS can be ameliorated, e.g., by decreasing the severity of the symptoms (such as the symptoms described above) associated with the, e.g., MS episode, or by lengthening the time period between the occurrence of episodes, e.g., by days, weeks, months, or years, where the episodes can be characterized by the flare-up and exacerbation of disease symptoms, or preventing or slowing the appearance of brain inflammatory lesions. See, e.g., Adams (1993) Principles of Neurology, page 777, for a description of a neurological inflammatory lesion.

The term “IFN-beta” or “IFN-β” as used herein refers to IFN-β or variants thereof, sometimes referred to as IFN-β-like polypeptides. Human IFN-β variants, which may be naturally occurring (e.g., allelic variants that occur at the IFN-β locus) or recombinantly produced, have amino acid sequences that are the same as, similar to, or substantially similar to the mature native IFN-β sequence. Fragments of IFN-β or truncated forms of IFN-β that retain their activity are also encompassed. These biologically active fragments or truncated forms of IFN-β are generated by removing amino acid residues from the full-length IFN-β amino acid sequence using recombinant DNA techniques well known in the art. IFN-β polypeptides may be glycosylated (IFN-β-1a) or unglycosylated (IFN-β-1b), as it has been reported in the literature that both the glycosylated and unglycosylated IFN-βs show qualitatively similar specific activities and that, therefore, the glycosyl moieties are not involved in and do not contribute to the biological activity of IFN-β.

The IFN-β variants encompassed herein include muteins of the mature native IFN-β sequence, wherein one or more cysteine residues that are not essential to biological activity have been deliberately deleted or replaced with other amino acids to eliminate sites for either intermolecular crosslinking or incorrect intramolecular disulfide bond formation. IFN-β variants of this type include those containing a glycine, valine, alanine, leucine, isoleucine, tyrosine, phenylalanine, histidine, tryptophan, serine, threonine, or methionine substituted for the cysteine found at amino acid 17 of the mature native amino acid sequence. Serine and threonine are the more preferred replacements because of their chemical analogy to cysteine. Serine substitutions are most preferred. In one embodiment, the cysteine found at amino acid 17 of the mature native sequence is replaced with serine. Cysteine 17 may also be deleted using methods known in the art (see, for example, U.S. Pat. No. 4,588,584, herein incorporated by reference), resulting in a mature IFN-β mutein that is one amino acid shorter than the mature native IFN-β. See also, as examples, U.S. Pat. Nos. 4,530,787; 4,572,798; and 4,588,585. Thus, IFN-β variants with one or more mutations that improve, for example, their pharmaceutical utility are also encompassed by the present invention.

The skilled artisan will appreciate that additional changes can be introduced by mutation into the nucleotide sequences encoding IFN-β, thereby leading to changes in the IFN-β amino acid sequence, without altering the biological activity of the interferon. Thus, an isolated nucleic acid molecule encoding an IFN-β variant having a sequence that differs from the amino acid sequence for the mature native IFN-β can be created by introducing one or more nucleotide substitutions, additions, or deletions into the corresponding nucleotide sequence disclosed herein, such that one or more amino acid substitutions, additions or deletions are introduced into the encoded IFN-β. Mutations can be introduced by standard techniques, such as site-directed mutagenesis and PCR-mediated mutagenesis. Such IFN-β variants are also encompassed by the present invention.

For example, conservative amino acid substitutions may be made at one or more predicted, preferably nonessential amino acid residues. A “nonessential” amino acid residue is a residue that can be altered from the wild-type sequence of IFN-β without altering its biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Such substitutions would not be made for conserved amino acid residues, or for amino acid residues residing within a conserved motif.

Alternatively, variant IFN-β nucleotide sequences can be made by introducing mutations randomly along all or part of an IFN-β coding sequence, such as by saturation mutagenesis, and the resultant mutants can be screened for IFN-β biological activity to identify mutants that retain activity. Following mutagenesis, the encoded protein can be expressed recombinantly, and the activity of the protein can be determined using standard assay techniques described herein.

Biologically active variants of IFN-β will generally have at least 80%, more preferably about 90% to about 95% or more, and most preferably about 96% to about 99% or more amino acid sequence identity to the amino acid sequence of mature native IFN-β, which serves as the basis for comparison. By “sequence identity” is intended the same amino acid residues are found within the variant polypeptide and the polypeptide molecule that serves as a reference when a specified, contiguous segment of the amino acid sequence of the variant is aligned and compared to the amino acid sequence of the reference molecule.

For purposes of optimal alignment of the two sequences for the purposes of sequence identity determination, the contiguous segment of the amino acid sequence of the variant may have additional amino acid residues or deleted amino acid residues with respect to the amino acid sequence of the reference molecule. The contiguous segment used for comparison to the reference amino acid sequence will comprise at least 20 contiguous amino acid residues. Corrections for increased sequence identity associated with inclusion of gaps in the variant's amino acid sequence can be made by assigning gap penalties. Methods of Sequence alignment are well known in the art.

Thus, the determination of percent identity between any two sequences can be accomplished using a mathematical algorithm. One preferred, non-limiting example of a mathematical algorithm utilized for the comparison of sequences is the algorithm of Myers and Miller (1988) Comput. Appl. Biosci. 4:11-7. Such an algorithm is utilized in the ALIGN program (version 2.0), which is part of the GCG alignment software package. A PAM120 weight residue table, a gap length penalty of 12, and a gap penalty of 4 can be used with the ALIGN program when comparing amino acid sequences. Another preferred, non-limiting example of a mathematical algorithm for use in comparing two sequences is the algorithm of Karlin and Altschul (1990) Proc. Natl. Acad. Sci. USA 90:5873-5877, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403-410. BLAST amino acid sequence searches can be performed with the XBLAST program, score=50, wordlength=3, to obtain amino acid sequence similar to the polypeptide of interest. To obtain gapped alignments for comparison purposes, gapped BLAST can be utilized as described in Altschul et al. (1997) Nucleic Acids Res. 25:3389-3402. Alternatively, PSI-BLAST can be used to perform an integrated search that detects distant relationships between molecules. See Altschul et al. (1997) supra. When utilizing BLAST, gapped BLAST, or PSI-BLAST programs, the default parameters can be used. See http://www.ncbi.nlm.nih.gov. Also see the ALIGN program (Dayhoff (1978) in Atlas of Protein Sequence and Structure 5:Suppl. 3, National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Comput. Appl. Biosci. 4:11-17.

Biologically active IFN-β variants encompassed by the invention also include IFN-β polypeptides that have covalently linked with, for example, polyethylene glycol (PEG) or albumin. These covalent hybrid IFN-β molecules possess certain desirable pharmaceutical properties such as an extended serum half-life after administration to a patient. Methods for creating PEG-IFN adducts involve chemical modification of monomethoxypolethylene glycol to create an activated compound that will react with IFN-β. Methods for making and using PEG-linked polypeptides are described, for example in Delgado et al. (1992) Crit. Rev. Ther. Drug. Carrier Syst. 9:249-304. Methods for creating albumin fusion polypeptides involve fusion of the coding sequences for the polypeptide of interest (e.g., IFN-β) and albumin and are described in U.S. Pat. No. 5,876,969, herein incorporated by reference.

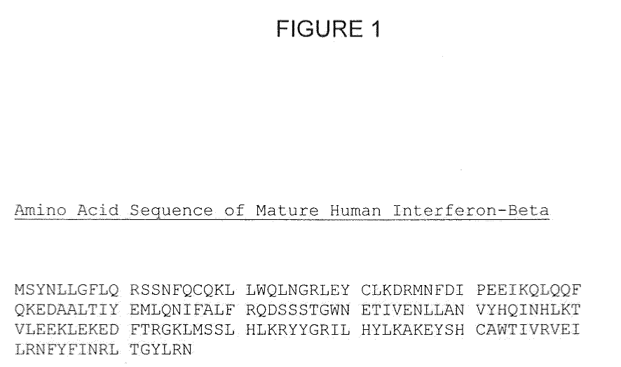

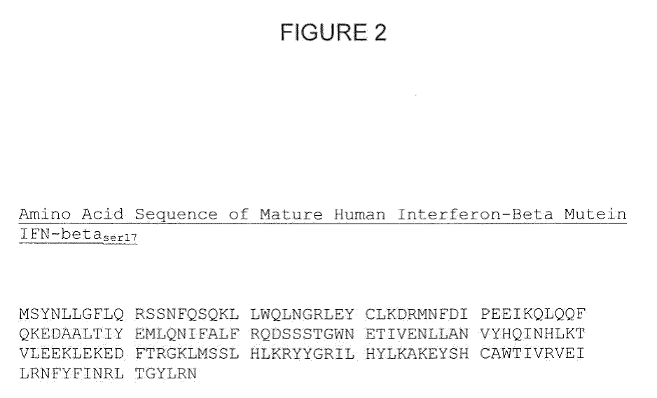

Biologically active variants of IFN-β encompassed by the invention should retain IFN-β activities, particularly the ability to bind to IFN-β receptors. In some embodiments, the IFN-β variant retains at least about 25%, about 50%, about 75%, about 85%, about 90%, about 95%, about 98%, about 99% or more of the biologically activity of the polypeptides whose amino acid sequences are given in FIG. 1 or 2. IFN-β variants whose activity is increased in comparison with the activity of the polypeptides shown in FIG. 1 or 2 are also encompassed. The biological activity of IFN-β variants can be measured by any method known in the art. Examples of such assays can be found in Fellous et al. (1982) Proc. Natl. Acad. Sci USA 79:3082-3086; Czerniecki et al. (1984) J. Virol. 49(2):490-496; Mark et al. (1984) Proc. Nall Acad. Sci. USA 81:5662-5666; Branca et al. (1981) Nature 277:221-223; Williams et al. (1979) Nature 282:582-586; Herberman et al. (1979) Nature 277:221-223; Anderson et al. (1982) J. Biol. Chew. 257(19):11301-11304.

The IFN-β for use in the methods of the invention can be from any animal species including, but not limited to, avian, canine, bovine, porcine, equine, and human. Preferably, the IFN-β is human IFN-β, more preferably is recombinantly produced human IFN-β, in either its glycosylated or unglycosylated form.

Non-limiting examples of IFN-β polypeptides and IFN-β variant polypeptides encompassed by the invention are set forth in Nagata et al. (1980) Nature 284:316-320; Goeddel et al. (1980) Nature 287:411-416; Yelverton et al. (1981) Nucleic Acids Res. 9:731-741; Streuli et al. (1981) Proc. Natl. Acad. Sci. U.S.A. 78:2848-2852; EP028033B1, and EP109748131. See also U.S. Pat. Nos. 4,518,584; 4,569,908; 4,588,585; 4,738,844; 4,753,795; 4,769,233; 4,793,995; 4,914,033; 4,959,314; 5,545,723; and 5,814,485. These disclosures are herein incorporated by reference. These citations also provide guidance regarding residues and regions of the IFN-β polypeptide that can be altered without the loss of biological activity.

In one embodiment of the present invention, the IFN-β used in the dosing regimens disclosed herein is the mature native human IFN-β polypeptide (FIG. 1), In another embodiment, the IFN-β in these formulations is the mature human IFN-β polypeptide wherein the cysteine found at amino acid 17 of the mature native sequence is replaced with serine as discussed above (FIG. 2; a mutein referred to herein as mature human IFN-β_{Ser17}). See U.S. Pat. No. 4,588,585, herein incorporated by reference. However, the present invention encompasses other embodiments where the IFN-β within the stabilized pharmaceutical formulation is any biologically active IFN-β polypeptide or variant as described elsewhere herein.

In some embodiments of the present invention, the IFN-β is recombinantly produced. By “recombinantly produced IFN-β” is intended IFN-β that has comparable biological activity to mature native IFN-β and that has been prepared by recombinant DNA techniques. IFN-β can be produced by culturing a host cell transformed with an expression vector comprising a nucleotide sequence that encodes an IFN-β polypeptide. The host cell is one that can transcribe the nucleotide sequence and produce the desired protein, and can be prokaryotic (for example, E. coli) or eukaryotic (for example a yeast, insect, or mammalian cell). Examples of recombinant production of IFN-β are given in Mantei et al. (1982) Nature 297:128; Ohno et al. (1982) Nucleic Acids Res. 10:967; Smith et al. (1983) Mol. Cell. Biol. 3:2156, and U.S. Pat. Nos. 4,462,940, 5,702,699, and 5,814,485; herein incorporated by reference. See also U.S. Pat. No. 5,795,779, where IFN-β-1a is recombinantly produced in Chinese hamster ovary (CHO) cells; herein incorporated by reference. Human interferon genes have been cloned using recombinant DNA (“rDNA”) technology and have been expressed in E. coli (Nagola et al. (1980) Nature 284:316; Goeddel et al. (1980) Nature 287:411; Yelverton et al. (1981) Nuc. Acid Res. 9:731; Streuli et al. (1981) Proc. Natl. Acad. Sci. U.S.A. 78:2848). Alternatively, IFN-β can be produced by a transgenic animal or plant that has been genetically engineered to express the IFN-β protein of interest in accordance with methods known in the art.

Proteins or polypeptides that exhibit native interferon-beta-like properties may also be produced with rDNA technology by extracting poly-A-rich 12S messenger RNA from virally induced human cells, synthesizing double-stranded cDNA using the mRNA as a template, introducing the cDNA into an appropriate cloning vector, transforming suitable microorganisms with the vector, harvesting the microorganisms, and extracting the interferon-beta therefrom. See, for example, European Patent Application Nos. 28033 (published May 6, 1981); 32134 (published Jul. 15, 1981); and 34307 (published Aug. 26, 1981), which describe various methods for the production of interferon-beta employing rDNA techniques.

Alternatively. IFN-β can be synthesized chemically, by any of several techniques that are known to those skilled in the peptide art. See, for example, Li et al. (1983) Proc. Natl. Acad. Sci. USA 80:2216-2220, Steward and Young (1984) Solid Phase Peptide Synthesis (Pierce Chemical Company, Rockford, Ill.), and Baraney and Merrifield (1980) The Peptides: Analysis, Synthesis, Biology, ed. Gross and Meinhofer, Vol. 2 (Academic Press, New York, 1980), pp. 3-254, discussing solid-phase peptide synthesis techniques; and Bodansky (1984) Principles of Peptide Synthesis (Springer-Verlag, Berlin) and Gross and Meinhofer, eds. (1980) The Peptides: Analysis, Synthesis, Biology, Vol. 1 (Academic Press, New York), discussing classical solution synthesis. IFN-β can also be chemically prepared by the method of simultaneous multiple peptide synthesis. See, for example, Houghten (1984) Proc. Natl. Acad. Sci. USA 82:5131-5135; and U.S. Pat. No. 4,631,211.

IFN-beta or biologically active variant thereof is formulated into pharmaceutical compositions for use in the methods of the invention. In this manner, a pharmaceutically acceptable carrier may be used in combination with the interferon and other components in the pharmaceutical composition. By “pharmaceutically acceptable carrier” is intended a carrier or diluent that is conventionally used in the art to facilitate the storage, administration, and/or the desired effect of the therapeutic ingredients. A carrier may also reduce any undesirable side effects of the therapeutic agent, i.e., IFN-beta or biologically active variant thereof. A suitable carrier should be stable, i.e., incapable of reacting with other ingredients in the formulation. It should not produce significant local or systemic adverse effect in recipients at the dosages and concentrations employed for therapy. Such carriers are generally known in the art. Suitable carriers for this invention are those conventionally used large stable macromolecules such as albumin, gelatin, collagen, polysaccharide, monosaccarides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), heparin alginate, and the like. Slow-release carriers, such as hyaluronic acid, may also be suitable. Stabilizers, such as trehalose, thioglycerol, and dithiothreitol (DTT), may also be added. Other acceptable components in the composition include, but are not limited to, buffers that enhance isotonicity such as water, saline, phosphate, citrate, succinate, acetic acid, and other organic acids or their salts.

Preferred pharmaceutical compositions may incorporate buffers having reduced local pain and irritation resulting from injection. Such buffers include, but are not limited to, low-phosphate buffers and succinate buffers. The pharmaceutical composition may additionally comprise a solubilizing compound that is capable of enhancing the solubility of IFN-beta or biologically active variant thereof.

For the purposes of this invention, the pharmaceutical composition comprising IFN-beta or biologically active variant thereof should be formulated in a unit dosage and in an injectable form such as solution, suspension, or emulsion. It can also be in the form of lyophilized powder, which can be converted into solution, suspension, or emulsion before intramuscular administration. The pharmaceutical composition may be sterilized by membrane filtration, which also removes aggregates, and stored in unit-dose or multi-dose containers such as sealed vials or ampules.

The method for formulating a pharmaceutical composition is generally known in the art. A thorough discussion of formulation and selection of pharmaceutically acceptable carriers, stabilizers, and isomolytes can be found in Remington's Pharmaceutical Sciences (18^{th} ed.; Mack Pub. Co.: Eaton, Pa. 1990), herein incorporated by reference.

Pharmaceutical compositions comprising IFN-beta or biologically active variant thereof are known in the art and include, but are not limited to, those disclosed in U.S. Pat. Nos. 5,183,746; 5,795,779; and 5,814,485. Also see copending U.S. Provisional Application No. 60/246,456, entitled “Stabilized Interferon Compositions,” filed Nov. 7, 2000; copending U.S. application Ser. No. 09/677,643, entitled “Stabilized Liquid Polypeptide-Containing Pharmaceutical Compositions,” filed Oct. 3, 2000; and copending U.S. Provisional Application No. 60/282,614, entitled “LISA-Free Formulations of Interferon-Beta,” filed Apr. 9, 2001; all of which are herein incorporated by reference.

Thus liquid, lyophilized, or spray-dried compositions comprising IFN-beta or biologically active variant thereof that are known in the art may be prepared as an aqueous or nonaqueous solution or suspension for subsequent administration to a subject in accordance with the methods of the invention. Each of these compositions will comprise IFN-beta or biologically active variant thereof as a therapeutically or prophylactically active component. By “therapeutically or prophylactically active component” is intended the IFN-beta or variant thereof is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

Effective treatment of MS in a subject using the methods of the invention can be examined in several alternative ways including, for example, EDSS (extended disability status scale) score, Functional Composite Score, cognitive testing, appearance of exacerbations, or MRI. Satisfying any of the following criteria evidences effective treatment.

The EDSS is a means to grade clinical impairment due to MS (Kurtzke (1983) Neurology 33:1444). Eight functional systems are evaluated for the type and severity of neurologic impairment. Briefly, prior to treatment, impairment in the following systems is evaluated: pyramidal, cerebellar, brainstem, sensory, bowel and bladder, visual, cerebral, and other. Follow-up scores are obtained at defined intervals. The scale ranges from 0 (normal) to 10 (death due to MS). An increase of one full step (or a one-half step at the higher baseline EDSS scores) defines disease progression in the context of the present invention (Kurtzke (1994) Ann. Neurol. 36:573-79, Goodkin (1991) Neurology. 41:332.).

Exacerbations are defined as the appearance of a new symptom that is attributable to MS and accompanied by an appropriate new neurologic abnormality (IFN-β MS Study Group). In addition, the exacerbation must last at least 24 hours and be preceded by stability or improvement for at least 30 days. Standard neurological examinations result in the exacerbations being classified as either mild, moderate, or severe according to changes in a Neurological Rating Scale (Sipe et al. (1984) Neurology 34:1368), changes in EDSS score or evaluating physician opinion. An annual exacerbation rate and proportion of exacerbation-free patients are determined. Therapy is deemed to be effective if there is a statistically significant difference in the rate or proportion of exacerbation-free patients between the treated group and the placebo group for either of these measurements. In addition, time to first exacerbation in patients with clinically isolated syndromes suggestive of MS and exacerbation duration and severity may also be measured. A measure of effectiveness of therapy in this regard is a statistically significant difference in the time to first exacerbation or duration and severity in the treated group compared to control group.

MRI can be used to measure active lesions using gadolinium-DTPA-enhanced T₁-weighted imaging (McDonald et al. (1994) Ann. Neurol. 36:14) or the location and extent of lesions using T₂-weighted techniques. Briefly, baseline MRIs are obtained. The same imaging plane and patient position are used for each subsequent study. Areas of lesions are outlined and summed slice by slice for total lesion area. Three analyses may be done: evidence of new lesions, rate of appearance of active or new lesions, and change in lesion area (Paty et al. (1993) Neurology 43:665). Improvement due to therapy is established when there is a statistically significant improvement in an individual patient compared to baseline or in a treated group versus a placebo group.

The following examples are offered by way of illustration and not by way of limitation.

### EXPERIMENTAL

### Example 1

### Pilot Clinical Trial Design Intended to Measure the Efficacy and Safety of a New Interferon-Beta Dosing Regimen

A pilot clinical trial is undertaken to measure the efficacy and safety of a new interferon-beta dosing regimen. Two dosing arms are included: Interferon-beta-1a at 6 MIU (30 ucg) administered intramuscularly once per week plus placebo administered once per week, versus interferon-beta at 6-12 MIU (30-60 ucg) administered intramuscularly twice weekly. A sample size of n=300-500 patients per arm is used. The duration of the study is 2 years, with a 1-year interim safety and efficacy analysis. The primary endpoint is time-to-confirmed disease progression or treatment failure as measured by EDSS or Multiple Sclerosis Functional Composite Score (Rudick (2001) Neurology 56(10): 1324-1330.

Secondary endpoints include relapse rate-related endpoints and MRI measurement-related endpoints. Tertiary endpoints include cognitive function-related endpoints and quality of life-related endpoints. Major safety endpoints include liver function, hematologic function, neutralizing antibody development, and injection site reactions.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Subheadings in the specification document are included solely for ease of review of the document and are not intended to be a limitation on the contents of the document in any way.

Although the foregoing invention has been described in some detail by way of illustration and example for purposes of clarity of understanding, it will be obvious that certain changes and modifications may be practiced within the scope of the present invention.

That which is claimed:

1. A method for treating multiple sclerosis in a subject in need thereof, said method comprising administering to said subject a therapeutically effective dose of about 9 MIU to about 30 MIU of a human interferon-beta mutein (hIFN-βₛₑᵣ₁₇), wherein said therapeutically effective dose is administered two times per week or three times per week by intramuscular injection.

2. The method of claim 1, wherein said therapeutically effective dose is in the range of about 9 MIU to about 12 MIU per injection.

3. The method of claim 1, wherein said therapeutically effective dose is in the range of about 11 MIU to about 13 MIU per injection.

4. The method of claim 1, wherein said therapeutically effective dose is in the range of about 13 MIU to about 15 MIU per injection.

5. The method of claim 1, wherein said therapeutically effective dose is administered intramuscularly two times per week.

6. The method of claim 1, wherein said hIFN-β_{Ser17} is recombinantly produced.

7. The method of claim 6, wherein said hIFN-β_{Ser17} is glycosylated.

8. The method of claim 6, wherein said hIFN-β_{Ser17} is unglycosylated.

9. The method of claim 1, wherein said multiple sclerosis is relapsing remitting multiple sclerosis.

10. The method of claim 9, wherein the frequency of exacerbations exhibited by said subject is decreased relative to the frequency of exacerbations in the absence of said method of treatment.

11. The method of claim 9, wherein the severity of exacerbations exhibited by said subject is decreased relative to the severity of exacerbations exhibited in the absence of said method of treatment.

12. The method of claim 9, wherein the rate of disease progression in said subject is slowed relative to the rate of disease progression in the absence of said method of treatment.

13. The method of claim 9, wherein the degree of brain inflammation is decreased relative to the degree of brain inflammation in the absence of said method of treatment.

14. The method of claim 1, wherein said intramuscular administration comprises administering said therapeutically effective dose of hIFN-β_{Ser17} into a muscle of a thigh, an upper arm, or a hip.

15. The method of claim 1, wherein said multiple sclerosis is secondary-progressive multiple sclerosis.


SEQUENCE LISTING (2 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 166 aa, Artificial Sequence
  Mutein of human interferon-beta
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN